(19) United States
(12) Patent Application Publication
(10) Pub. No.: US 2022/0315905 A1
(43) Pub. Date: Oct. 6, 2022

(54) Enzymatic Reaction Composition, Method for Increasing Amount of Adenosine Triphosphate (ATP) in Enzymatic Reaction and Application Thereof

(71) Applicants: Sup Yin TSANG, Hong Kong (CN); Bioright Worldwide Company Limited, Tortola (VG)

(72) Inventors: Sup Yin Tsang, Hong Kong (CN); Wing Keung Poon, Hong Kong (CN); Yau Lung Siu, Hong Kong (CN); Kam Chun Lo, Hong Kong (CN); Jun Wang, Hong Kong (CN)

(21) Appl. No.: 17/618,091

(22) PCT Filed: Jun. 1, 2020
(86) PCT No.: PCT/CN2020/093704

§ 371 Date: Dec. 10, 2021

(30) Foreign Application Priority Data
Jun. 11, 2019 (CN) 201910502226.4

(51) Int. Cl. C12N 9/12; C12N 15/70

(52) U.S. Cl. CPC C12N 9/1229; C12N 15/70

(57) ABSTRACT

Provided are an enzymatic reaction composition, a method for increasing the amount of adenosine triphosphate (ATP) in an enzymatic reaction, and a method for synthesizing amino acids or derivatives thereof, polypeptides, enzymes or proteins by using ATP. In the method, a first enzyme or enzyme group for producing adenosine monophosphate (AMP) is added during the enzymatic reaction so as to additionally increase the amount of ATP.

### CROSS-REFERENCE TO RELATED APPLICATIONS

This application is the United States national phase of International Application No. PCT/CN2020/093704 filed Jun. 1, 2020, and claims priority to Chinese Patent Application No. 201910502226.4 filed Jun. 11, 2019, the disclosures of which are hereby incorporated by reference in their entirety.

The Sequence Listing associated with this application is filed in electronic format via EFS-Web and is hereby incorporated by reference into the specification in its entirety. The name of the text file containing the Sequence Listing is 2107659_ST25.txt. The size of the text file is 58,428 bytes, and the text file was created on Dec. 10, 2021.

### BACKGROUND OF THE INVENTION

### Field of the Invention

The present invention relates to the field of biochemistry, in particular to a biochemical reaction using adenosine triphosphate (ATP), especially an enzymatic reaction using adenosine triphosphate, an enzymatic reaction composition and a method for increasing the content of adenosine triphosphate (ATP) in the enzymatic reaction.

### Description of Related Art

An adenosine triphosphate (ATP) is a high-energy compound composed of three linked phosphate groups, ribose and adenine. Adenosine triphosphate (ATP) is one of the most important nicotinamide adenine dincleotide in the organism, providing energy for metabolism and providing phosphate groups and adenosine groups and so on. The important components of adenosine triphosphate (ATP) are three linked α, β and γ-phosphate groups, wherein the α-phosphate group is connected to adenosine, and the β and γ phosphate groups are high-energy phosphate bonds. The compound only containing α-phosphate group is adenosine monophosphate (AMP); the compound containing both α and β phosphate groups is adenosine diphosphate (ADP); and the compound containing all the α, β and γ phosphate groups is adenosine triphosphate (ATP). In enzymatic reactions involving adenosine triphosphate (ATP) (for example, using adenosine triphosphate (ATP) as energy source or/and phosphate group as substrate), the synthetase may use one or two high-energy phosphate bonds in adenosine triphosphate (ATP) or/and use phosphate groups as substrates. After the reaction is completed, ATP is converted into adenosine diphosphate (ADP) or adenosine monophosphate (AMP) etc. Adenosine triphosphate (ATP) is also used as a substrate in biological reactions; and the synthetase can use the adenosine portion of adenosine triphosphate (ATP) as a substrate, which transfers its adenosine group to other compounds, producing the by-product such as nicotinamide adenine dinucleotide with monophosphoric acid or pyrophosphoric acid.

Polypeptide synthesis and phosphorylation production through enzymatic reactions are important means in modern biotechnology. Compared with chemical synthesis methods, the enzymatic production has many advantages including: in the synthesis process, enzymatic production does not use organic chemicals that are harmful to human or toxic to the environment, and generally does not produce unwanted by-products during the reaction, which is thus advantageous for purification. However, the current enzymatic process using adenosine triphosphate (ATP) has not been widely used due to cost and other reasons.

There is still a need for further improved enzymatic processes or enzymatic reactions using adenosine triphosphate (ATP).

### SUMMARY OF THE INVENTION

The invention provides an enzymatic reaction composition, an enzymatic reaction, a method for increasing the content of adenosine triphosphate (ATP) in an enzymatic reaction, and use thereof.

Specifically, the present invention provides:

(1) A method for increasing an amount of adenosine triphosphate (ATP) in an enzymatic reaction, wherein a first enzyme or enzyme group for producing adenosine monophosphate (AMP) and adenosine are added during the enzymatic reaction to additionally increase the amount of adenosine triphosphate (ATP), wherein a reaction substrate of the enzymatic reaction comprises adenosine triphosphate (ATP) or a salt thereof.

(2) The method according to any of the above, wherein the method further comprises adding a second enzyme or enzyme group responsible for adenosine triphosphate (ATP) regeneration at the same time as, before or after adding the first enzyme or enzyme group.

(3) The method according to any of the above, wherein a third enzyme or enzyme group is added at the same time as, before or after adding the first enzyme or enzyme group and/or adding the second enzyme or enzyme group.

(4) The method according to any of the above, wherein the first enzyme or enzyme group comprises adenosine kinase (AK).

(5) The method according to any of the above, wherein the reaction substrate further comprises at least one of polyphosphoric acid or a salt thereof and an auxiliary ion, wherein the auxiliary ion is preferably at least one of magnesium ion, sodium ion, potassium ion and chloride ion, and more preferably at least one of magnesium ion and potassium ion; the auxiliary ion may be in the form of an inorganic salt or an organic salt, preferably at least one of magnesium chloride hexahydrate, sodium chloride, manganese chloride, magnesium sulfate, and potassium carbonate, and more preferably at least one of magnesium chloride hexahydrate, sodium chloride, and potassium carbonate.

(6) The method according to any of the above, wherein the second enzyme or enzyme group comprises at least one of polyphosphate:AMP phosphotransferase (PAP), polyphosphokinase (PPK) and adenylate kinase (ADK); and optionally, the third enzyme or enzyme group comprises creatine kinase (CK), glutamate kinase (GK), nicotinamide adenine dincleotide kinase (NK), and/or other enzymes or enzyme groups for performing phosphorylation, phosphate group(s) transfer or polypeptide synthesis of amino acids, peptides or proteins in which adenosine triphosphate (ATP) serves as one of the substrates.

(7) The method according to any of the above, wherein the enzymatic reaction comprises at least one of the following:

(i) the first enzyme or enzyme group comprises adenosine kinase (AK), the reaction substrate comprises adenosine, polyphosphoric acid and adenosine triphosphate (ATP), and the reaction product comprises adenosine monophosphate (AMP) and adenosine diphosphate (ADP);
(ii) the first enzyme or enzyme group comprises adenosine kinase (AK), and the second enzyme or enzyme group comprises polyphosphate:AMP phosphotransferase (PAP), and the reaction substrate comprises adenosine monophosphate (AMP) and polyphosphoric acid, and the reaction product contains adenosine diphosphate (ADP) and polyphosphoric acid;
(iii) the first enzyme or enzyme group comprises adenosine kinase (AK), and the second enzyme or enzyme group comprises adenylate kinase (ADK), and the reaction substrate comprises adenosine diphosphate (ADP) and polyphosphoric acid, and the reaction product contains adenosine monophosphate (AMP) and adenosine triphosphate (ATP);
(iv) the first enzyme or enzyme group comprises adenosine kinase (AK), and the second enzyme or enzyme group comprises polyphosphokinase (PPK), and the reaction substrate comprises adenosine diphosphate (ADP) and polyphosphoric acid, and the reaction product contains adenosine triphosphate (ATP) and polyphosphoric acid; and
(v) the first enzyme or enzyme group comprises adenosine kinase (AK), and the second enzyme or enzyme group comprises polyphosphate:AMP phosphotransferase (PAP), polyphosphokinase (PPK) and adenylate kinase (ADK), and the third enzyme or enzyme group comprises creatine kinase (CK), the reaction substrate comprises creatine and adenosine triphosphate (ATP), and the reaction product comprises Phosphocreatine, adenosine diphosphate (ADP) and polyphosphoric acid; the third enzyme or enzyme group also comprises glutamate kinase (GK), the reaction substrate contains glutamate and adenosine triphosphate (ATP), and the reaction product contains glutamate 5-phosphate, adenosine diphosphate (ADP)) and polyphosphoric acid; the third enzyme or enzyme group may also comprise nicotinamide adenine dincleotide kinase (NK), the reaction substrate comprises nicotinamide adenine dincleotide and adenosine triphosphate (ATP), and the reaction product comprises oxidized nicotinamide adenine dinucleotide phosphate, adenosine diphosphate (ADP) and polyphosphoric acid; the third enzyme or enzyme group may also comprise other enzymes or enzyme groups for performing phosphorylation, phosphotransfer or peptide synthesis of amino acids, nucleic acids, peptides or proteins in which adenosine triphosphate (ATP) serves as one of the enzymatic reaction substrates;
(vi) use of all or at least one of polyphosphate:AMP phosphotransferase (PAP), adenylate kinase (ADK) and phosphokinase, and adenosine kinase, with adenosine triphosphate (ATP) as the reaction substrate, to carry out reaction simultaneously in the same reaction system in the manner of mixing, parallel or series, or carry out reaction separately in different reaction systems;
(vii) only use of all or at least one of polyphosphate:AMP phosphotransferase (PAP), adenosine kinase (AK) and polyphosphokinase (PPK), with adenosine triphosphate (ATP) as the reaction substrate, to carry out reaction simultaneously in the same reaction systems in the manner of mixing, parallel or series, or carry out reaction separately in different reaction systems; and
(viii) only use of adenosine kinase, with adenosine as a substrate, to produce adenosine monophosphate (AMP), thereby newly adding adenosine triphosphate (ATP).

(8) The method according to any of the above, wherein the second enzyme or enzyme group regenerates adenosine diphosphate (ADP) and adenosine triphosphate (ATP) from adenosine monophosphate (AMP) and adenosine diphosphate (ADP), respectively; and
optionally, the first enzyme or enzyme group synthesizes adenosine monophosphate (AMP) from adenosine.

(9) The method according to any of the above, the enzymatic reaction comprises synthesizing adenosine monophosphate (AMP) using adenosine and adenosine triphosphate (ATP) as substrates.

(10) The method according to any of the above, wherein the reaction substrate further comprises creatine or a hydrate thereof, sodium glutamate or a hydrate thereof, and/or nicotinamide adenine dincleotide, etc.

(11) The method according to any of the above, wherein the first enzyme or enzyme group to be added is determined according to the level of the degradation product of adenosine triphosphate (ATP) produced in the enzymatic reaction, and wherein the degradation product is preferably adenosine diphosphate (ADP), adenosine monophosphate (AMP) and/or adenosine.

(12) The method according to any of the above, wherein the first enzyme or enzyme group, or the second enzyme or enzyme group, or the third enzyme or enzyme group is added in the form of purified or non-purified cells lysates, liquid enzymes, immobilized cells or immobilized enzymes.

(13) The method according to any of the above, wherein the conditions of the enzymatic reaction are as follows: a temperature of 28-40 degrees Celsius, preferably 30-38 degrees Celsius, and more preferably 33-37 degrees Celsius; and a pH value of 5-9, preferably 6-8.5, and more preferably 7-7.75.

(14) An enzymatic reaction composition comprising a substrate and a first enzyme or enzyme group for producing adenosine monophosphate (AMP), wherein the substrate comprises adenosine triphosphate (ATP) or a salt thereof, and adenosine.

(15) The enzymatic reaction composition according to any of the above, wherein the substrate further comprises at least one of polyphosphoric acid or a salt thereof, an auxiliary ion, and creatine or a hydrate thereof; wherein the auxiliary ion is preferably at least one of magnesium ion, sodium ion, potassium ion and chloride ion, and more preferably at least one of magnesium ion and potassium ion; the auxiliary ion may be in the form of an inorganic salt or an organic salt, preferably at least one of magnesium chloride hexahydrate, sodium chloride, manganese chloride, magnesium sulfate, and potassium carbonate, and more preferably at least one of magnesium chloride hexahydrate, sodium chloride, and potassium carbonate.

(16) The enzymatic reaction composition according to any of the above, wherein the enzymatic reaction composition further comprises a second enzyme or enzyme group and optionally a third enzyme or enzyme group.

(17) The enzymatic reaction composition according to any of the above, wherein the first enzyme or enzyme group comprises adenosine kinase (AK), preferably, the third enzyme or enzyme group, when present, comprises creatine kinase (CK), glutamate kinase (GK), nicotinamide adenine dincleotide kinase (NK), or other enzymes or enzyme groups for performing phosphorylation, phosphate group transfer or polypeptide synthesis of amino acids, peptides or proteins in which adenosine triphosphate (ATP) serves as one of the enzymatic reaction substrates, and preferably, the second enzyme or enzyme group comprises at least one of polyphosphate:AMP phosphotransferase (PAP), polyphosphate kinase (PPK), and adenylate kinase (ADK).

(18) The enzymatic reaction composition according to any of the above, wherein the first enzyme or enzyme group, the second enzyme or enzyme group and the third enzyme or enzyme group are contained in the enzymatic reaction composition in the form of purified or non-purified cells lysates, liquid enzymes, immobilized cells or immobilized enzymes.

(19) The enzymatic reaction composition according to any of the above, wherein the polyphosphate:AMP phosphotransferase (PAP), adenosine kinase (AK), polyphosphokinase (PPK) and adenylate kinase (ADK) and creatine kinases are each respectively recombinant protein, and are expressed in E. coli respectively or in combination.

(20) A method for phosphorylation or phosphate group transfer of amino acids, nucleic acids, peptides or proteins using adenosine triphosphate (ATP) as a substrate, including any of the above methods.

(21) The method according to any of the above, wherein the amino acid, nucleic acid, peptide or protein is creatine, glutamic acid, nicotinamide adenine dincleotide, and the like.

The present invention has the following advantages and positive effects:

1. The present invention can utilize not only polyphosphoric acid as a substrate, but also inexpensive adenosine as a reaction substrate, so that the amount of adenosine triphosphate (ATP) among the reactants can be additionally increased and/or the by-products in the reaction such as adenosine phosphate (AMP) and adenosine diphosphate (ADP) can be regenerated into adenosine triphosphate (ATP), resulting in that more adenosine triphosphate (ATP) are produced. Therefore, in the enzymatic reaction, the amount of adenosine triphosphate (ATP) increases with the reaction time, rather than being decreased. However, the existing adenosine triphosphate (ATP) regeneration technology can only maintain a stable amount of adenosine triphosphate (ATP) under optimal conditions. Adenosine triphosphate (ATP) is expensive and due to limitation in the cost. Limited amount of ATP is allowed to add in industrial production in order to control production costs. When the concentration of adenosine triphosphate (ATP) in the reaction is too low, the reaction efficiency will not be ideal, therefore, the side effect serves as wasting production capacity and further increase the degree of complexity of purification process and eventually dismish the recovery percentage of the final product. According to the present invention, the amount of adenosine triphosphate (ATP) in the enzymatic reaction will gradiently increase along with the number of reaction cycles. As the result, the total reaction efficiency will gradually increase along with the reaction time, and the related reaction speed will also increase accordingly.
2. The present invention utilizes adenosine, low-cost and common compound, to synthesize adenosine monophosphate (AMP) and later adenosine triphosphate (ATP), which can further reduce the production cost of existing reactions that require the use of adenosine triphosphate (ATP) as the required substrate.
3. Adenosine triphosphate (ATP), adenosine diphosphate (ADP) and adenosine monophosphate (AMP)-degrading enzymes have high enzymatic activity within E. coli cells. When adenosine monophosphate (AMP) is degraded into adenosine, adenosine triphosphate (ATP) essentially abolishs in the reaction, which eventually leads to the cessation of the reaction. In the present invention, the adenosine produced after hydrolysis can be re-synthesized into adenosine monophosphate (AMP), and then re-converted into adenosine triphosphate (ATP). Therefore, the enzymes can be used even without going through the purification process, which greatly saves the huge investment cost and time due to the purification procedure and enable the enzymatic production process to be industrially implemented in terms of cost and production capacity.

However, the current method of regeneration using adenosine triphosphate (ATP) usually requires purification process of related enzymes in the protein. This method is extremely expensive and will put a significant financial pressure on production costs. Therefore, the enzymatic production technology has not been popularized.

### DESCRIPTION OF THE INVENTION

The present invention will be further explained through the detailed description of embodiments below, but this is not a limitation to the present invention. Those skilled in the art can make various modifications or improvements according to the basic idea of the present invention, but as long as not deviating from the basic idea of the present invention, they fall within the scope of the present invention.

As mentioned above, compared with the chemical synthesis method, the enzymatic process has many advantages. However, the enzymatic process also has its shortcomings: in occasion the enzymatic reactions have to rely on the requirement of using expensive adenosine triphosphate (ATP) in biological reactions, which puts a significant financial pressure on production costs. At present, the polyphosphoric acid is generally used as a substrate to regenerate adenosine diphosphate (ADP) or adenosine monophosphate (AMP) into adenosine triphosphate (ATP) by phosphate transfer, or glucose is used as the raw material for adenosine triphosphate (ATP) to reduce the cost of adenosine triphosphate (ATP). Although such adenosine triphosphate (ATP) regeneration technology can reduce the cost pressure of adenosine triphosphate (ATP), it suffers from other difficulties. Firstly, in order to reduce the total production cost, the amount of the adenosine triphosphate (ATP) used in the system had to be restricted, which leads to underachieve efficiency of the enzymatic synthesis reaction. Secondly, the recombinant enzyme expressed by Escherichia coli as a host contains a large amount of enzymes that capable to hydrolyze both adenosine triphosphate (ATP), adenosine diphosphate (ADP) and adenosine monophosphate (AMP) into degraded by-product. These hydrolases have high activities and can hydrolyze adenosine triphosphate (ATP), adenosine diphosphate (ADP), and adenosine monophosphate (AMP) to adenosine monophosphate (AMP) and adenosine in a short time. Adenosine triphosphate (ATP) is degraded into adenosine and then disappears irreversibly. Therefore, if the industry is intended to use adenosine triphosphate (ATP) regeneration technology to reduce cost, purification of recombinant enzyme is the only move but such process is uneconomical

In order to solve at least one of the above-mentioned problems, the inventors of the present invention have conducted a lot of in-depth theoretical research and experimental exploration to produce a larger amount of adenosine triphosphate (ATP) using low-cost adenosine as a substrate for the production of adenosine monophosphate (AMP) through an adenosine triphosphate (ATP) regeneration and/or production reaction system. Therefore, during the enzymatic reaction process, the amount of adenosine triphosphate (ATP) increases with the reaction time, thereby increasing the reaction efficiency and reducing the cost.

In one aspect, the present invention provides an enzymatic reaction composition comprising a substrate and a first enzyme or enzyme group for producing adenosine monophosphate (AMP), wherein the substrate comprises adenosine and adenosine triphosphate (ATP) or a salt thereof.

Optionally, the substrate further comprises polyphosphoric acid or a salt thereof, preferably the sodium salt of polyphosphoric acid. The polyphosphoric acid may have a degree of polymerization of 3-20,000; preferably, a degree of polymerization of 3-7,000, and more preferably 3-75.

Optionally, the enzymatic reaction composition may further include auxiliary ions. The auxiliary ion may be selected from at least one of magnesium ion, sodium ion, potassium ion, and chloride ion, and more preferably at least one of magnesium ion and potassium ion. The auxiliary ion may be in the form of an inorganic salt or an organic salt, and is preferably at least one of magnesium chloride hexahydrate, sodium chloride, manganese chloride, magnesium sulfate and potassium carbonate, and more preferably at least one of magnesium chloride hexahydrate, sodium chloride and potassium carbonate.

In order to utilize the enzymatic reaction composition capable of rapidly adding new adenosine triphosphate (ATP) for a reaction synthesis (including use of adenosine triphosphate (ATP) as a substrate for phosphoric acid exchange and synthesis, as well as use of it as energy for polypeptide synthesis or phosphoric acid exchange), the reaction also contains the substrate required for the synthesis reaction. Herein, the enzymatic synthesis of Phosphocreatine is illustrated as an example, and the reaction substrate includes creatine and adenosine triphosphate (ATP) with the auxiliary ion of magnesium which is supplied in the form of magnesium chloride hexahydrate.

In addition, it is well known in the art that the enzymatic reaction composition may also contain other additives, for example, pH adjusting agents, such as buffers/salts, preferably sodium phosphate buffer, potassium phosphate buffer and tris(hydroxymethyl)aminomethane (known as TRIS) buffer liquid, more preferably sodium phosphate buffer and TRIS buffer. The pH adjusting agent may have a concentration of 0.001 M-1 M, preferably 0.01 M-0.5 M, and more preferably 0.05 M-0.3 M.

Optionally, the enzymatic reaction composition further comprises a second enzyme or enzyme group and optionally a third enzyme or enzyme group. Preferably, the first enzyme or enzyme group comprises adenosine kinase (AK). Preferably, the third enzyme or enzyme group, if present, includes at least one of creatine kinase (CK), glutamate kinase (GK) and nicotinamide adenine dincleotide kinase (NK).

Preferably, the second enzyme or enzyme group comprises at least one of polyphosphate:AMP phosphotransferase (PAP), polyphosphate kinase (PPK) and adenylate kinase (ADK).

Optionally, the first enzyme or enzyme group, the second enzyme or enzyme group, and the third enzyme or enzyme group contained in the enzymatic reaction composition may be used in the form of purified or non-purified cells lysates, liquid enzymes, immobilized cells or immobilized enzymes. For the immobilized cells or the immobilized enzymes, they may be prepared according to the method described in Chinese Patent No. CN1982445B and the carrier in the patent can be used. For example, the carrier is a porous organic foam material with open pores, and the shape of the carrier includes granular, block, column, sheet or strip shape. Preferably, the porous organic foam material is melamine sponge.

Hereinafter, an enzymatic reaction composition refers to a mixture of substrates that is required of carrying out a biochemical reaction with the present of an enzyme or an enzyme group to catalyze the reaction to form product. The reaction products may include one or more of adenosine monophosphate (AMP), adenosine diphosphate (ADP), adenosine triphosphate (ATP), polyphosphoric acid, phosphocreatine, glutamate 5-phosphate, and nicotinamide adenine dincleotide phosphate.

Here, the first enzyme or enzyme group, the second enzyme or enzyme group, and the third enzyme or enzyme group may each be respectively expressed in recombinant protein or in combination. The expression host may include Escherichia coli and yeast cells. In this way, cells or fragments thereof containing recombinant protein can be used to perform the enzymatic reaction. For example, the cells may be E. coli cells or yeast cells. The expression host can be Escherichia coli, yeast, Bacillus and so on, which may express the recombinant protein in the methods commonly used in molecular biology. The enzyme or enzyme group can be used as cells solution, lysates, supernatant or purified enzyme, or may be made with its corresponding carrier nto the immobilized cells or immobilized enzymes to perform enzyme or enzyme group reactions.

In another aspect, the present invention also provides a method for increasing the amount of adenosine triphosphate (ATP) in an enzymatic reaction, which comprises adding a first enzyme or enzyme group that produces adenosine monophosphate (AMP) from adenosine during the enzymatic reaction to additionally increase the amount of adenosine triphosphate (ATP), wherein the reaction substrate of the enzymatic reaction contains adenosine triphosphate (ATP) or a salt thereof.

The method may also include adding a second enzyme or enzyme group responsible for adenosine triphosphate (ATP) regeneration at the same time as, before or after adding the first enzyme or enzyme group.

Optionally, a third enzyme or enzyme group may be added at the same time as, before or after adding the first enzyme or enzyme group and/or adding the second enzyme or enzyme group.

As mentioned above, the first enzyme or enzyme group may comprise adenosine kinase (AK). Optionally, the reaction substrate further comprises at least one of polyphosphoric acid or a salt thereof and auxiliary ions.

The auxiliary ion is preferably at least one of magnesium ion, sodium ion, potassium ion and chloride ion, and more preferably at least one of magnesium ion and potassium ion. The auxiliary ion may be in the form of an inorganic salt or an organic salt, preferably at least one of magnesium chloride hexahydrate, sodium chloride, manganese chloride, magnesium sulfate, and potassium carbonate, and more preferably at least one of magnesium chloride hexahydrate, sodium chloride, and potassium carbonate.

As described above, the second enzyme or enzyme group may comprise at least one of polyphosphate:AMP phosphotransferase (PAP), polyphosphate kinase (PPK), adenylate kinase (ADK) and creatine kinase.

Optionally, the enzymatic reaction includes at least one of the following:

(i) the first enzyme or enzyme group includes adenosine kinase (AK), the reaction substrate includes adenosine, polyphosphoric acid and adenosine triphosphate (ATP), and the reaction product includes adenosine monophosphate (AMP) and diphosphate adenosine (ADP);
(ii) the first enzyme or enzyme group includes adenosine kinase (AK), and the second enzyme or enzyme group includes polyphosphate:AMP phosphotransferase (PAP), and the reaction substrate includes adenosine monophosphate (AMP) and polyphosphoric acid, and the reaction product contains adenosine diphosphate (ADP) and polyphosphoric acid;
(iii) the first enzyme or enzyme group includes adenosine kinase (AK), and the second enzyme or enzyme group includes adenylate kinase (ADK), and the reaction substrate includes adenosine diphosphate (ADP) and polyphosphoric acid, and the reaction product contains adenosine monophosphate (AMP) and adenosine triphosphate (ATP);
(iv) the first enzyme or enzyme group includes adenosine kinase (AK), and the second enzyme or enzyme group includes polyphosphokinase (PPK), and the reaction substrate includes adenosine diphosphate (ADP) and polyphosphoric acid, and the reaction product contains adenosine triphosphate (ATP) and polyphosphoric acid; and
(v) the first enzyme or enzyme group includes adenosine kinase (AK), and the second enzyme or enzyme group includes polyphosphate:AMP phosphotransferase (PAP), polyphosphokinase (PPK) and adenylate kinase (ADK), and the third enzyme or enzyme group includes creatine kinase (CK), the reaction substrate includes creatine and adenosine triphosphate (ATP), and the reaction product includes phosphocreatine, adenosine diphosphate (ADP) and polyphosphoric acid; the third enzyme or enzyme group also contains glutamate kinase (GK), the reaction substrate contains glutamate and adenosine triphosphate (ATP), and the reaction product contains glutamate 5-phosphate, adenosine diphosphate (ADP) and polyphosphoric acid; the third enzyme or enzyme group may also include nicotinamide adenine dincleotide kinase (NK), the reaction substrate includes nicotinamide adenine dincleotide and adenosine triphosphate (ATP), and the reaction product includes oxidized nicotinamide adenine dinucleotide phosphate, adenosine diphosphate (ADP) and polyphosphoric acid; the third enzyme or enzyme group may also include other enzymes or enzyme groups for performing phosphorylation, phosphotransfer or polypeptide synthesis of amino acids, nucleic acids, peptides or proteins in which adenosine triphosphate (ATP) serves as one of the enzymatic reaction substrates.
(vi) use of all or at least one of polyphosphate:AMP phosphotransferase (PAP), adenylate kinase (ADK) and phosphokinase (PPK), and adenosine kinase (AK), with adenosine triphosphate (ATP) as the reaction substrate, to carry out reaction simultaneously in the same reaction system in the manner of mixing, parallel or series, or carry out reaction separately in different reaction systems;
(vii) only use of all or at least one of polyphosphate:AMP phosphotransferase (PAP), adenylate kinase (ADK) and phosphokinase (PPK), with adenosine triphosphate (ATP) as the reaction substrate, to carry out the reaction simultaneously in the same reaction systems in the manner of mixing, parallel or series, or carry out reaction separately in different reaction systems; and
(viii) only use of adenosine kinase (AK), with adenosine as a substrate, to produce adenosine monophosphate (AMP), thereby newly adding adenosine triphosphate (ATP).

In one embodiment, the second enzyme or enzyme group regenerates adenosine diphosphate (ADP) and adenosine triphosphate (ATP) from adenosine monophosphate (AMP) and adenosine diphosphate (ADP), respectively; and optionally, the first enzyme or enzyme group synthesizes adenosine monophosphate (AMP) from adenosine.

In one embodiment, the enzymatic reaction involves the synthesis of adenosine monophosphate (AMP) using adenosine and adenosine triphosphate (ATP) as substrates.

In one embodiment, the reaction substrate may also include creatine or a hydrate thereof.

In one embodiment, the first enzyme or enzyme group to be added is determined according to the degradant level of adenosine triphosphate (ATP) produced in the enzymatic reaction, and the degradants are preferably adenosine diphosphate (ADP), adenosine monophosphate (AMP) and/or adenosine.

In one embodiment, the first enzyme or enzyme group, or the second enzyme or enzyme group, or the third enzyme or enzyme group is added in the form of purified or non-purified cells lysates, liquid enzymes, immobilized cells or immobilized enzymes.

Optionally, the enzymatic reaction can be performed at a temperature of 28-40 degrees Celsius, preferably 30-38 degrees Celsius, and more preferably 33-37 degrees Celsius; and at a pH value of 5-9, preferably 6-8.5, and more preferably 7-7.75.

In this way, the method of the present invention can utilize by-products in the reaction system (such as adenosine, adenosine diphosphate (ADP) or adenosine monophosphate (AMP)) or additionally added adenosine as a substrate to rapidly produce additional adenosine triphosphate (ATP) or regenerate adenosine triphosphate (ATP), thereby producing a greater amount of adenosine triphosphate (ATP). Therefore, in the enzymatic reaction, the amount of adenosine triphosphate (ATP) increases with the reaction time instead of decreasing.

Optionally, the enzymatic reaction composition may further include at least one of polyphosphoric acid or salts thereof, auxiliary ions, salts, and creatine or hydrates thereof.

Optionally, the reaction product of the enzymatic reaction comprises one or more of adenosine monophosphate (AMP), adenosine diphosphate (ADP), adenosine triphosphate (ATP), polyphosphoric acid and phosphocreatine.

In the enzymatic reaction, the polyphosphate:AMP phosphotransferase (PAP), adenosine kinase (AK), polyphosphate kinase (PPK) and adenylate kinase (ADK), creatine kinase (CK), glutamate kinase (GK) and nicotinamide adenine dincleotide kinase (NK) are each resepectively a recombinant protein, and are expressed in E. coli separately or in combination.

In yet another aspect, the present invention also provides a method for phosphorylation or phosphotransfer of amino acids, nucleic acids, peptides or proteins using adenosine triphosphate (ATP) as a substrate, including any of the above methods. Therefore, the above methods can be used to synthesize amino acids or derivatives thereof, nucleic acids, peptides, proteins or derivatives thereof.

In a specific example, the method involves a peptide synthesis or phosphorylation reaction using adenosine triphosphate (ATP) as a substrate, specifically including adding the substrate, and auxiliary ion or salt required for the reaction to perform the synthesis reaction, and adding synthase for rapidly producing additional adenosine triphosphate (ATP) and rapidly regenerating adenosine triphosphate (ATP), substrates, and magnesium ion to simultaneously perform the rapid production and regeneration reaction of adenosine triphosphate (ATP) under suitable reaction conditions for polypeptide synthesis or phosphorylation.

Preferably, the amino acid, peptide, nucleic acid, protein or derivative thereof is creatine, glutamic acid, nicotinamide adenine dincleotide and the like.

In order to perform the enzymatic reaction, the immobilized cells and/or the immobilized enzymes install in an immobilization reaction device to perform an immobilization reaction. For example, the immobilization reaction can be carried out according to the steps described in Chinese Patent Application Publication No. CN106032520A.

The immobilization reaction device may include a columnar reactor with an inlet and an outlet, a reaction regulating tank, a high-flow water pump, a pH regulating device and a pH detecting electrode, a stirring device, and a pH regulating tank.

The content of the present invention will be further explained or illustrated by way of the following examples, but these examples should not be construed as limiting the scope of protection of the present invention.

### EXAMPLES

If the specific conditions are not indicated in the following examples, they shall be carried out according to the conventional conditions or the conditions recommended by the manufacturers. Unless otherwise specified, the percentages are percentages by weight.

The materials and equipment used in the following examples are described below:

Reaction control tank: GeneHarbor (Hong Kong) Biotechnologies, Ltd., BR-1L;

Flow-adjustable type suction pump: SURGEFLO Company, FL-32;

PH control device: GeneHarbor (Hong Kong) Biotechnologies, Ltd., AR-1;

Creatine monohydrate: Jiangsu Yuanyang Chemical Co., Ltd.;

Adenosine: Zhejiang Chengyi Pharmaceutical;

Adenosine triphosphate disodium salt: Kaiping Qianniu Pharmaceutical Company;

Polyphosphoric acid: Xilong Chemical Co., Ltd.;

Sodium Glutamate: Ajinomoto Red Bowl MSG;

Nicotinamide adenine dincleotide: Roche Inc., USA.

### Example 1: Preparation of Creatine Kinase (CK)

The sequences of PCR primers of the creatine kinase were designed based on the Chinese Patent Application Publication No. CN102808006, specifically:

Upstream primer CPK1:
(SEQ NO. 1)
5′-ctgaccggatccatgccgttcggtaacacccacaac-3′

wherein, the BamHI restriction site is underlined.

Downstream primer CPK2:
(SEQ NO. 2)
5′-tatgcggaattcttacttctgggcggggatcatgtc-3′

wherein, the EcoRI restriction site is underlined.

According to the method described in Chinese Patent Application

Publication No. CN102808006, the total RNA from mouse skeletal muscle was extracted, and cDNA was prepared by reverse transcription. Using the mouse skeletal muscle cDNA as a model, the creatine kinase gene was obtained by PCR amplification of the above primers and ligated to pGEX-2T (purchased from GE Healthcare, USA), thus obtaining pGEX-2T(+)-CK (SEQ NO. 11), which was transformed into E. coli BL21 (DE3) to obtain a recombinant expression strain of creatine kinase.

A single colony of the above-mentioned strains was added into 4 mL of LB medium (containing 100 ug/ml ampicillin), and cultivated in a shaker at 37° C. and 200 rpm for 16 hours as primary seed. Afterwards, the resultant was added into 100 mL of LB medium (containing 100 ug/ml ampicillin) at 1% inoculation ratio, and cultured in a shaker at 37° C. and 200 rpm for 10 hours as secondary seed. Afterwards, the secondary seed was added into 60 L of LB medium (containing 100 ug/ml ampicillin) at 1% inoculation ratio and cultured in a 100 L fermenter. The initial fermentation conditions were 37° C., 200 rpm, and pH 7.0. The fermentation was carried out for 9 hours, then IPTG was added to a final concentration of 1 mM, and the fermentation was completed after 20 hours. The fermentation broth was centrifuged at 12,500 rpm at 4° C. for 10 minutes to obtain 1.42 kg of E. coli cells with the overexpression of creatine kinase. The obtained E. coli cells with the overexpression of creatine kinase were prepared into an enzyme solution comprising 0.2 g cells per ml of the enzyme solution. According to the method for determination of Phosphocreatine content described in Chinese Patent Application Publication No. CN102808006, the enzyme activity of the cells was detected, and the enzyme activity was about 2.1 U/g.

### Example 2: Preparation of Polyphosphate:AMP Transferase (PAP)

PCR primers were designed based on the sequence of polyphosphate:AMP phosphotransferase, specifically:

Upstream primer PAP1:
(SEQ NO. 3)
5′-ctgaccggatccatgttcgaatccgcggaagttggc-3

wherein, the BamHI restriction site is underlined.

Downstream primer PAP2:
(SEQ NO. 4)
5′-tatgcgaagcttttacttgtccttcttgtacgccgcctc-3′

wherein, the EcoRI restriction site is underlined.

DNA from Pseudomonas aeruginosa PAO1-VE13 AGY71676 was used as a substrate, and the polyphosphate:AMP transferase gene was obtained by PCR amplification of the above primers. The PCR products were treated with restriction enzymes BamH I and EcoRI and ligated to pGEX-2T (purchased from GE Healthcare, USA), thus obtaining pGEX-2T(+)-PAP (SEQ NO. 12). This recombinant expression vector was transformed into Escherichia coli HB101 to obtain a recombinant expression strain of polyphosphate:AMP phosphotransferase.

A single colony of the above-mentioned strains was added into 4 mL of LB medium (containing 100 ug/ml ampicillin), and cultivated in a shaker at 37° C. and 200 rpm for 16 hours as primary seed. Afterwards, the resultant was added into 100 mL of LB medium (containing 100 ug/ml ampicillin) at 1% inoculation ratio, and cultured in a shaker at 37° C. and 200 rpm for 10 hours as secondary seed. Afterwards, the secondary seed was added into 60L of LB medium (containing 100 ug/ml ampicillin) at 1% inoculation ratio and cultured in a 100 L fermenter. The initial fermentation conditions were 37° C., 200 rpm, and pH 7.0. The fermentation was carried out for 9 hours, then isopropyl thiogalactoside (IPTG) was added to a final concentration of 1 mM, and the fermentation was completed after 20 hours. The fermentation broth was centrifuged at 12,500 rpm for 10 minutes at 4° C. to obtain 1.18 kg of E. coli cells with overexpression of polyphosphate:AMP phosphotransferase. The obtained E. coli cells with overexpression of polyphosphate:AMP phosphotransferase were prepared into an enzyme solution comprising 0.2 g cells per ml of the enzyme solution. According to its enzymatic reaction, the cells's enzyme activity was tested, and the cells's enzyme activity was about 1.1 U/g.

### Example 3: Preparation of Adenylate Kinase (AK)

PCR primers were designed based on the sequence of adenylate kinase, specifically:

Upstream primer AK1:
(SEQ NO. 5)
5′-ctgaccggatccatggcagtcgattcctccaactcg-3

wherein, the BamHI restriction site is underlined.

Downstream primer AK2:
(SEQ NO. 6)
5′-tatgcggaattcttaacacggaagtgaagtgaagct-3′

wherein, the EcoRI restriction site is underlined.

DNA from Salmonella enterica subsp. enterica serovar ATCC 700720 (ATCC, USA) was used as the substrate, and PCR amplification was performed with the above primers to obtain the adenylate kinase gene. The PCR products were treated with restriction endonucleases BamHI and EcoRI and ligated to pGEX-2T (purchased from GE Healthcare, USA), thus obtaining pGEX-2T(+)-ADK (SEQ NO. 13). This recombinant expression vector was transformed into Escherichia coli HB101 to obtain a recombinant expression strain of adenylate kinase.

A single colony of the above-mentioned strains was added into 4 mL of LB medium (containing 100 ug/ml ampicillin), and cultivated in a shaker at 37° C. and 200 rpm for 16 hours as primary seed. Afterwards, the resultant was added into 100 mL of LB medium (containing 100 ug/ml ampicillin) at 1% inoculation ratio, and cultured in a shaker at 37° C. and 200 rpm for 10 hours as secondary seed. Afterwards, the secondary seed was added into 60 L of LB medium (containing 100 ug/ml ampicillin) at 1% inoculation ratio and cultured in a 100 L fermenter. The initial fermentation conditions were 37° C., 200 rpm, and pH 7.0. The fermentation was carried out for 9 hours, then IPTG was added to a final concentration of 1 mM, and the fermentation was completed after 20 hours. The fermentation broth was centrifuged at 12,500 rpm for 10 minutes at 4° C. to obtain 1.18 kg of E. coli cells with the overexpression of adenylate kinase. The obtained E. coli cells with the overexpression of adenylate kinase were prepared into an enzyme solution comprising 0.2 g cells per ml of the enzyme solution. The enzyme activity of the cells was detected according to the enzymatic reaction, and the enzyme activity was about 0.08 U/g.

### Example 4: Preparation of Polyphosphokinase (PPK)

PCR primers were designed based on the sequence of polyphosphokinase, specifically:

Upstream primer PPK-1:
(SEQ NO. 7)
5′-ctgaccggatccatgagcaagtccgacgacgacgag-3

wherein, the BamHI restriction site is underlined.

Downstream primer PPK-2:
(SEQ NO. 8)
5′-tatgcggaattcttaccgcgccaaccgcccatcttc-3′

wherein, the EcoRI restriction site is underlined.

DNA from C. crescentus NA1000YP_002518902 was used as the substrate and PCR amplification was performed with the above primers to obtain the phosphokinase gene. The PCR product was treated with restriction enzymes BamHI and EcoRI and ligated to pGEX-2T (purchased from GE Healthcare, USA), thus obtaining pGEX-2T(+)-PPK (SEQ NO. 14). This recombinant expression vector was transformed into Escherichia coli HB101 to obtain a recombinant expression strain of polyphosphokinase.

A single colony of the above-mentioned strains was added into 4 mL of LB medium (containing 100 ug/ml ampicillin), and cultivated in a shaker at 37° C. and 200 rpm for 16 hours as primary seed. Afterwards, the resultant was added to 100 ml of LB medium (containing 100 ug/ml ampicillin) at 1% inoculation ratio and cultured in a shaker at 37° C. and 200 rpm for 10 hours as secondary seed. Afterwards, the secondary seed was added to 60 L of LB medium (containing 100 ug/ml ampicillin) at 1% inoculation ratio and cultured in a 100 L fermenter. The initial fermentation conditions were 37° C., 200 rpm, and pH 7.0. The fermentation was carried out for 9 hours, then IPTG was added to a final concentration of 1 mM, and the fermentation was completed after 20 hours. The fermentation broth was centrifuged at 12,500 rpm for 10 minutes at 4° C. to obtain 1.73 kg of E. coli cells with the overexpression of polyphosphokinase. The obtained E. coli cells with the overexpression of polyphosphokinase were prepared into an enzyme solution comprising 0.2 g of cells per ml of the enzyme solution. The enzyme activity of the cells was detected according to the enzymatic reaction, and the enzyme activity was about 0.03 U/g.

### Example 5: Preparation of Adenosine Kinase (ADK)

PCR primers were designed based on the sequence of adenosine kinase, specifically:

Upstream primer ADK1:
(SEQ NO. 9)
5′-ctgaccggatccatgaatatcattttgatgggttta-3

wherein, the BamHI restriction site is underlined.

Downstream primer ADK2:
(SEQ NO. 10)
5′-tatgcggaattcttacaaatgatctaaaatatcaat-3′

wherein, the EcoRI restriction site is underlined.

DNA from Mycobacterium smegmatis MC2 155 was used as the substrate, and the adenosine kinase gene sequence was obtained by PCR amplification with the above primers. The PCR product was treated with restriction enzymes BamHI and EcoRI, and the resultant gene sequence was ligated to pGEX-2T (purchased from GE Healthcare, USA), thus obtaining pGEX-2T(+)-AK (SEQ NO.15), which was transformed into E. coli BL21(DE3) to obtain a recombinant expression strain of adenosine kinase.

A single colony of the above-mentioned strains was added into 4 mL of LB medium (containing 100 ug/ml ampicillin), and cultivated in a shaker at 37° C. and 200 rpm for 16 hours as primary seed. Afterwards, the resultant was added to 100 ml of LB medium (containing 100 ug/ml ampicillin) at 1% inoculation ratio and cultured in a shaker at 37° C. and 200 rpm for 10 hours as secondary seed. Afterwards, the secondary seed was added to 60 L of LB medium (containing 100 ug/ml ampicillin) at 1% inoculation ratio and cultured in a 100 L fermenter. The initial fermentation conditions were 37° C., 200 rpm, and pH 7.0. The fermentation was carried out for 9 hours, then IPTG was added to a final concentration of 1 mM, and the fermentation was completed after 20 hours. The fermentation broth was centrifuged at 12,500 rpm for 10 minutes at 4° C. to obtain 1.2 kg of E. coli cells with the overexpression of adenosine kinase. The obtained E. coli cells with the overexpression of adenosine kinase were suspended in 50 mM Tris-HCl hydrochloric acid buffer (pH 7.5) at a ratio of 1:5. Then, the bacterial cells were lysed with ultrasound wave. The lysates were centrifuged (10° C., 12,500 rpm, 15 minutes) and the supernatant was collected as adenosine kinase solution. According to the enzymatic reaction, the cells's enzyme activity was tested, and the enzyme activity was about 0.08 EU/g.

### Example 6: Preparation of Glutamate Kinase (GK)

PCR primers were designed based on the sequence of glutamate kinase, specifically:

Upstream primer GKl:
(SEQ NO. 16)
5′-ctgaccggatccatgcgggacaaggtgactggcgcg-3′

wherein, the BamHI restriction site is underlined.

Downstream primer GK2:
(SEQ NO. 17)
5′-tatgcggaattcttagaccagaaccagattgtcgcg-3′

wherein, the EcoRI restriction site is underlined.

Pseudomonas aeruginosa was used as the substrate, and the adenosine kinase gene sequence was obtained by PCR amplification with the above primers. The PCR product was treated with restriction enzymes BamHI and EcoRI, and the resultant gene sequence was ligated to pGEX-2T (purchased from GE In Healthcare, USA), thus obtaining pGEX-2T(+)-GK (SEQ NO.20), which was then transformed into E. coli BL21(DE3) to obtain a recombinant expression strain of adenosine kinase.

A single colony of the above-mentioned strains was added into 4 mL of LB medium (containing 100 ug/ml ampicillin), and cultivated in a shaker at 37° C. and 200 rpm for 16 hours as primary seed. Afterwards, the resultant was added to 100 ml of LB medium (containing 100 ug/ml ampicillin) at 1% inoculation ratio, and cultured in a shaker at 37° C. and 200 rpm for 10 hours as secondary seed. Afterwards, the secondary seed was added to 60 L of LB medium (containing 100 ug/ml ampicillin) at 1% inoculation ratio and cultured in a 100 L fermenter. The initial fermentation conditions were 37° C., 200 rpm, and pH 7.0. The fermentation was carried out for 9 hours, then IPTG was added to a final concentration of 1 mM, and the fermentation was completed after 20 hours. The fermentation broth was centrifuged at 12,500 rpm for 10 minutes at 4° C. to obtain 1.2 kg of E. coli cells containing glutamate kinase. The obtained E. coli cells with the overexpression of glutamate kinase were suspended in 50 mM Tris-HCl hydrochloric acid buffer (pH 7.5) at a ratio of 1:5. Then, the bacterial cells were lysed with ultrasound wave. The lysates were centrifuged (10° C., 12,500 rpm, 15 minutes) and the supernatant was collected as glutamate kinase solution. According to the enzymatic reaction, the cells's enzyme activity was tested, and the enzyme activity was about 0.02 EU/g.

### Example 7: Preparation of Nicotinamide Adenine Dincleotide Kinase (NK)

PCR primers were designed based on the sequence of nicotinamide adenine dincleotide kinase, specifically:

Upstream primer NK1:
(SEQ NO. 18)
5′-ctgaccggatccatgcgggacaaggtgactggcgcg-3′

wherein, the BamHI restriction site is underlined.

Downstream primer NK2:
(SEQ NO. 19)
5′-tatgcggaattcttagaccagaaccagattgtcgcg-3′

wherein, the EcoRI restriction site is underlined.

Mycobacterium tuberculosis variant bovis BCG str. Tokyo 172 was used as a substrate and PCR amplification was performed with the above primers to obtain the sequence of nicotinamide adenine dincleotide kinase gene. The PCR product was treated with restriction enzymes BamHI and EcoRI, and the resultant gene sequence was ligated to pGEX-2T (purchased from GE Healthcare, USA), thus obtaining pGEX-2T(+)-NK (SEQ NO.21), which was transformed into E. coli BL21(DE3) to obtain a recombinant expression strain of nicotinamide adenine dincleotide kinase.

A single colony of the above-mentioned strains were added into 4 mL of LB medium (containing 100 ug/ml ampicillin), and cultivated in a shaker at 37° C. and 200 rpm for 16 hours as primary seed. Afterwards, the resultant was added to 100 mL of LB medium (containing 100 ug/ml ampicillin) at 1% inoculation ratio, and cultured in a shaker at 37° C. and 200 rpm for 10 hours as secondary seed. Afterwards, the secondary seed was added to 60 L of LB medium (containing 100 ug/ml ampicillin) at 1% inoculation ratio and cultured in a 100 L fermenter. The initial fermentation conditions were 37° C., 200 rpm, and pH 7.0. The fermentation was carried out for 9 hours, then IPTG was added to a final concentration of 1 mM, and the fermentation was completed after 20 hours. The fermentation broth was centrifuged at 12,500 rpm for 10 minutes at 4° C. to obtain 1.2 kg of E. coli cells with the overexpression of nicotinamide adenine dincleotide Kinase (NK). The obtained E. coli cells with the overexpression of nicotinamide adenine dincleotide kinase were suspended in 50 mM Tris-HCl hydrochloric acid buffer (pH 7.5) at a ratio of 1:5. Then, the bacterial cells were lysed with ultrasound wave. The lysates were centrifuged (10° C., 12,500 rpm, 15 minutes) and the supernatant was collected as nicotinamide adenine dincleotide kinase solution. According to the enzymatic reaction, the enzyme activity of the cells was tested, and the enzyme activity was about 0.045 EU/g.

### Example 8: Preparation of Phosphocreatine

According to the preparation methods of Examples 1-5, the genes of creatine kinase, polyphosphate:AMP phosphotransferase, adenylate kinase, polyphosphokinase, adenosine kinase and the recombinant enzyme overexpressed in E. coli cells expressing these genes respectively were obtained, and then fermented.

According to the method of Example 3 of Chinese Patent No. CN1982445B, a mixed immobilized Escherichia coli cells with the overexpression of creatine kinase, adenosine kinase, polyphosphate:AMP phosphotransferase, adenylate kinase and polyphosphokinase were prepared on a solid-phase carrier. The mixing weight ratio of the respective cells was substantially based on the corresponding enzyme activity. Table 1 below shows the wet weight of the respective cells after centrifugation. The shape of the carrier was a bar shape as follows: length 24 cm, width 5 cm, thickness 5 mm, and actual weight 46.3 g. The above-prepared carrier having the mixed immobilized E. coli cells was installed in an immobilized enzyme or immobilized cells reactor. The reactor was a cylinder made of organic glass with a height of 7 cm and a radius of 4.5 cm. A knife was used to trim off about 3 cm of the head and tail of the above-mentioned carrier at an inclination of 45°, and the remainder was tightly held and rolled into a homogeneous cylinder with a height of 5 cm and a radius of 4.5 cm, with a weight of 32.2 g. The cylinder was inserted into the reactor to a tightness meeting the level 3 standard described in Table 1 of Chinese Patent Application No. CN106032520A, and there was no gap between its side wall and the inner wall of the reactor. After the installation was completed, the installation procedures of other equipment were followed according to FIG. 1 of CN106032520A. The capacity of the reaction control tank was 1 L; the high-flow water pump was a flow-adjustable suction pump with a flow rate of 3 L/min; for the pH control device, 0.3 M sodium hydroxide solution was adopted to adjust the pH, and the flow rate of the dosing pump was 1 ml per minute. The substrates contained in the reaction solution were as follows: 8.94 g/L of creatine monohydrate, 3 g/L of adenosine triphosphate disodium salt, 16.4 g/L of magnesium chloride hexahydrate, 6 g/L of adenosine, and 13.3 g/L of polyphosphoric acid. After adding the above substrates, the resultant was stirred and dissolved with 1 liter of deionized water at about 45° C. until the reaction solution was clear, and the pH was adjusted to 8.5 with 2 M sodium hydroxide solution. 300 ml of the above reaction solution was added to the reaction control tank with the temperature being 37° C., the pH being adjusted to 8.35-8.60 and the flow rate of the high-flow water pump set at 3 L/min. When the reaction continued for 60 minutes, the content of Phosphocreatine of the product was 6.5 mM, and when the reaction lasted for 90 minutes, the concentration of Phosphocreatine in the product solution was 11.3 mM. At the end of the reaction for 120 minutes, the concentration of Phosphocreatine of the product solution was 15.7 mM.

TABLE 1

| Enzyme | Weight of cells (g) |
|---|---|
| Creatine kinase (CK) | 12.3 |
| Adenosine kinase (AK) | 11.5 |
| Polyphosphate: AMP | 12.2 |
| phosphotransferase (PAP) | |
| Adenylate kinase (ADK) | 13.2 |
| Polyphosphokinase (PPK2) | 9.7 |

### Example 9: Preparation of Glutamate 5-Phosphate

According to the preparation methods of Examples 2-6, the genes of polyphosphate:AMP phosphotransferase (PAP), adenylate kinase (ADK), polyphosphokinase (PPK), adenosine kinase (AK) and glutamate kinase (GK) and the E. coli cells with recombinant overexpression of these genes respectively were obtained, and then fermented.

According to the method of Example 3 of Chinese Patent No. CN1982445B, a mixed immobilized Escherichia coli cells with the overexpression of glutamate kinase, adenosine kinase, polyphosphate:AMP phosphotransferase, adenylate kinase and polyphosphokinase were prepared on a solid-phase carrier. The mixing weight ratio of the respective cells was substantially based on the corresponding enzyme activity. Table 2 below shows the wet weight of the respective cells after centrifugation. The shape of the carrier was a bar shape as follows: length 28 cm, width 5 cm, thickness 5 mm, and actual weight 48.1 g. The above-prepared carrier having the mixed immobilized E. coli cells was installed in an immobilized enzyme or immobilized cells reactor. The reactor was a cylinder made of organic glass with a height of 7 cm and a radius of 4.5 cm. A knife was used to trim off about 3 cm of the head and tail of the above-mentioned carrier at an inclination of 45°, and the remainder was tightly held and rolled into a homogeneous cylinder with a height of 5 cm and a radius of 4.5 cm, with a weight of 31.4 g. The cylinder was inserted into the reactor to a tightness meeting the level 3 standard described in Table 1 of Chinese Patent Application No. CN106032520A, and there was no gap between its side wall and the inner wall of the reactor. After the installation was completed, the installation procedures of other equipment were followed according to FIG. 1 of CN106032520A. The capacity of the reaction control tank was 1 L; the high-flow water pump was a flow-adjustable suction pump with a flow rate of 3 L/min; for the pH control device, 0.3 M sodium hydroxide solution was adopted to adjust the pH, and the flow rate of the dosing pump was 1 ml per minute. The substrates contained in the reaction solution were as follows: sodium glutamate 3.5 g/L, adenosine triphosphate disodium salt 3 g/L, magnesium chloride hexahydrate 16.4 g/L, adenosine 6 g/L, and polyphosphoric acid 13.3 g/L. After adding the above substrates, the resultant was stirred and dissolved with 1 liter of deionized water at about 30° C. until the reaction solution was clear, and the pH was adjusted to 7.5 with 2 M sodium hydroxide solution. 300 ml of the above reaction solution was added to the reaction control tank with the temperature being 32° C., the pH being adjusted to 7.25-7.5 and the flow of the high-flow water pump being 3 L/min. After 60 minutes of reaction, the concentration of the product solution glutamate 5-phosphate was 6.8 mM, and after 90 minutes of reaction, the concentration of the product solution glutamate 5-phosphate was 8.4 mM. At the end of the reaction for 120 minutes, the concentration of the product solution glutamate 5-phosphate was 9.8 mM.

TABLE 2

| Enzyme | Weight of cells (g) |
|---|---|
| Glutamate Kinase (GK) | 14.8 |
| Adenosine Kinase (AK) | 11.5 |
| Polyphosphate: AMP | 12.2 |
| phosphotransferase (PAP) | |
| Adenylate Kinase (ADK) | 13.2 |
| Polyphosphokinase (PPK2) | 9.7 |

### Example 10: Preparation of Oxidized Nicotinamide Adenine Dinucleotide Phosphate

According to the preparation methods of Examples 2-5 and 7, the genes of polyphosphate:AMP phosphotransferase, adenylate kinase, polyphosphokinase, adenosine kinase and nicotinamide adenine dincleotide kinase and the recombinant enzyme overexpressed in E. coli cells expressing these genes respectively were obtained, and then fermented.

According to the method of Example 3 of Chinese Patent No. CN1982445B, a mixed immobilized Escherichia coli cells with the overexpression of nicotinamide adenine dincleotide kinase (NK), adenosine kinase (AK), polyphosphate:AMP phosphotransferase (PAP), adenylate kinase (ADK) and polyphosphokinase (PPK) were prepared on a solid-phase carrier. The mixing weight ratio of the respective cells was substantially based on the corresponding enzyme activity. Table 3 below shows the wet weight of the respective cells after centrifugation. The shape of the carrier was a bar shape as follows: length 21 cm, width 5 cm, thickness 5 mm, and actual weight 40.8 g. The above-prepared carrier having the mixed immobilized E. coli cells was installed in an immobilized enzyme or immobilized cells reactor. The reactor was a cylinder made of organic glass with a height of 7 cm and a radius of 4.5 cm. A knife was used to trim off about 3 cm of the head and tail of the above-mentioned carrier at an inclination of 45°, and the remainder was tightly held and rolled into a homogeneous cylinder with a height of 5 cm and a radius of 4.5 cm, with a weight of 30.8 g. The cylinder was inserted into the reactor to a tightness meeting the level 3 standard described in Table 1 of Chinese Patent Application No. CN106032520A, and there was no gap between its side wall and the inner wall of the reactor. After the installation was completed, the installation procedures of other equipment were followed according to FIG. 1 of CN106032520A. The capacity of the reaction control tank was 1 L; the high-flow water pump was a flow-adjustable suction pump with a flow rate set at 3 L/min; for the pH control device, 0.3 M sodium hydroxide solution was adopted to adjust the pH, and the flow rate of the dosing pump was 1 ml per minute. The substrates contained in the reaction solution were as follows: oxidized nicotinamide adenine dinucleotide 20.3 g/L, adenosine triphosphate disodium salt 3 g/L, magnesium chloride hexahydrate 16.4 g/L, adenosine 6 g/L, and polyphosphoric acid 13.3 g /L. After adding the above substrates, the resultant was stirred and dissolved with 1 liter of deionized water at about 30° C. until the reaction solution was clear, and the pH was adjusted to 7.5 with 2 M sodium hydroxide solution. 300 ml of the above reaction solution was added to the reaction control tank with the temperature being 35° C., the pH being adjusted to 7.75-8, and the flow of the high-flow water pump being 3 L/min. After 60 minutes of reaction, the concentrtion of the oxidized nicotinamide adenine dinucleotide phosphate product solution was 7.2 mM; after 90 minutes of reaction, the concentration of the oxidized nicotinamide adenine dinucleotide phosphate product solution was 9.4 mM; and at the end of the reaction for 120 minutes, the concentration of the oxidized nicotinamide adenine dinucleotide phosphate product solution was 13.1 mM.

TABLE 3

| Enzyme | Weight of cells (g) |
|---|---|
| Nicotinamide adenine | 12.1 |
| dincleotide kinase (GK) | |
| Adenosine Kinase (AK) | 11.5 |
| Polyphosphate: AMP | 12.2 |
| phosphotransferase (PAP) | |
| Adenylate Kinase (ADK) | 13.2 |
| Polyphosphokinase (PPK2) | 9.7 |

### Example 11: Using Adenosine as a Substrate to Quickly Increase the Content of Adenosine Triphosphate (ATP)

According to the preparation methods of Examples 2-5, the genes of polyphosphate:AMP phosphotransferase (PAP), adenylate kinase (ADK), polyphosphokinase (PPK), adenosine kinase (AK) and adenosine kinase (AK) and the recombinant enzyme overexpressed in E. coli cells expressing these genes respectively were obtained, and then fermented.

According to the method of Example 3 of Chinese Patent No. CN1982445B, a mixed immobilized Escherichia coli cells with the overexpression of polyphosphate:AMP phosphotransferase (PAP), adenylate kinase (ADK), polyphosphokinase (PPK) and adenosine kinase (AK) were prepared on a solid-phase carrier. The mixing weight ratio of the respective cells was substantially based on the corresponding enzyme activity. Table 4 below shows the wet weight of the respective cells after centrifugation. The shape of the carrier was a bar shape as follows: length 18 cm, width 5 cm, thickness 5 mm, and actual weight 37.5 g. The above-prepared carrier having the mixed immobilized E. coli cells was installed in an immobilized enzyme or immobilized cells reactor. The reactor was a cylinder made of organic glass with a height of 7 cm and a radius of 4.5 cm. A knife was used to trim off about 3 cm of the head and tail of the above-mentioned carrier at an inclination of 45°, and the remainder was tightly held and rolled into a homogeneous cylinder with a height of 5 cm and a radius of 4.5 cm, with a weight of 27.2 g. The cylinder was inserted into the reactor to a tightness meeting the level 3 standard described in Table 1 of Chinese Patent Application No. CN106032520A, and there was no gap between its side wall and the inner wall of the reactor. After the installation was completed, the installation procedures of other equipment were followed according to FIG. 1 of CN106032520A. The capacity of the reaction control tank was 1 L; the high-flow water pump was a flow-adjustable suction pump with a flow rate of 1 L/min; for the pH control device, 0.3 M sodium hydroxide solution was adopted to adjust the pH, and the flow rate of the dosing pump was 1 ml per minute. The substrates contained in the reaction solution were as follows: adenosine 6 g/L, magnesium chloride hexahydrate 16.4 g/L, and polyphosphoric acid 13.3 g/L. After adding the above substrates, the resultant was stirred and dissolved with 1 liter of deionized water at about 45° C. until the reaction solution was clear, and the pH was adjusted to 7-7.5 with 5 M sodium hydroxide solution. 1 L of the above reaction solution was added to the reaction control tank, with the temperature being 37° C., the pH being adjusted to 7-7.5, and the flow rate of the high-flow water pump set at 2 L/min. The reaction lasted for 120 minutes, and the concentration of the product solution of adenosine triphosphate (ATP) was 20 mM.

Generally, a large amount of adenosine triphosphate (ATP) has been degraded to adenosine diphosphate (ADP), adenosine monophosphate (AMP) and adenosine at the very early stage of the above-mentioned reaction process, thereby being depleted. However, the adenosine triphosphate (ATP) concentration of the product solution obtained by the above method was 20 mM, and therefore, the content of adenosine triphosphate (ATP) was rapidly increased during the reaction.

TABLE 4

| Enzyme | Weight of cells (g) |
|---|---|
| Adenosine Kinase (AK) | 10.3 |
| Polyphosphate: AMP | 10.5 |
| phosphotransferase (PAP) | |
| Adenylate Kinase (ADK) | 11.2 |
| Polyphosphokinase (PPK2) | 10.4 |

### Example 12: Enzymatic Production of Phosphocreatine (Without AK, PAP, ADK and PPK)

According to the preparation method of Example 1, the gene of the creatine kinase and the E. coli cells expressing the gene were obtained and fermented. According to the method described in Example 3 of Chinese Patent CN1982445B, the immobilized cells were prepared on a carrier using the obtained recombinantly expressed E. coli cells of creatine kinase having wet weight of 35 g. The shape of the carrier was a bar shape as follows: length 27 cm, width 5 cm, thickness 5 mm, and actual weight 58.4 g. The substrates contained in the reaction solution were as follows: creatine monohydrate 8.94 g/L, adenosine triphosphate disodium salt 3 g/L, magnesium chloride hexahydrate 16.4 g/L, adenosine 6 g/L, and polyphosphoric acid 13.3 g/L. After adding the above substrates, the resultant was stirred and dissolved with 1 liter of deionized water at about 45° C. until the reaction solution was clear, and the pH was adjusted to 8.5 with 2 M sodium hydroxide solution. 300 ml of the above reaction solution was added to the reaction control tank, with the temperature being 37° C., the pH being adjusted to 8.35-8.60, and the flow of the high-flow water pump being 3 L/min. The reaction lasted for 60 minutes. At the very early stage of the reaction process, a large amount of adenosine triphosphate (ATP) was degraded to adenosine diphosphate (ADP), adenosine monophosphate (AMP) and adenosine and thus could not be used. The concentration of the Phosphocreatine product solution was 0.8 mM, with a total weight of 0.05 g.

### Example 13: Enzymatic Production of Phosphocreatine (Without AK)

According to the preparation methods of Examples 1-2 and 4-5, creatine kinase (CK), polyphosphate:AMP phosphotransferase (PAP), adenylate kinase (ADK), polyphosphokinase (PPK) and the recombinant enzyme overexpressed in E. coli cells respectively were obtained and fermented. According to the method described in Example 3 of Chinese Patent CN1982445B, the mixed immobilized cells of the E. coli cells expressing polyphosphate:AMP phosphotransferase, the E. coli cells expressing adenylate kinase, the E. coli cells expressing polyphosphokinase and the E. coli cells expressing creatine kinase were prepared on a carrier. Table 5 below shows the wet weight of the respective cells after centrifugation.

The mixing weight ratio of the respective cells was substantially based on the corresponding enzyme activity. The shape of the carrier was a bar as follows: 34 cm long, 5 cm wide, and 5 mm thick, with an actual weight of 46.6 g. The substrates contained in the reaction solution were as follows: 8.94 g/L of creatine monohydrate, 3 g/L of adenosine triphosphate disodium salt, 16.4 g/L of magnesium chloride hexahydrate, 6 g/L of adenosine, and 13.3 g/L of polyphosphoric acid. After adding the above substrates, the resultant was stirred and dissolved with 1 liter of deionized water at about 45° C. until the reaction solution was clear, and the pH was adjusted to 8.5 with 2 M sodium hydroxide solution. 300 ml of the above reaction solution was added to the reaction control tank, with the temperature being 37° C., the pH being adjusted to 8.35-8.60, and the flow rate of the high-flow water pump set at 3 L/min. The reaction continued for 60 minutes. Since adenosine kinase (AK) in the combination of adenosine triphosphate (ATP) enzymatic regeneration and production was not included, adenosine triphosphate (ATP) was slowly degraded to adenosine over time course during the reaction. When the reaction was completed, the reaction solution contained the product of Phosphocreatine, a large amount of adenosine and a small amount of adenine, in which the concentration of the Phosphocreatine product solution was 3.8 mM, with a total amount of 0.24 g.

TABLE 5

| Enzyme | Weight of cells (g) |
|---|---|
| Creatine kinase (CK) | 17.4 |
| Polyphosphate: AMP | 17.5 |
| phosphotransferase (PAP) | |
| Adenylate Kinase (ADK) | 14.5 |
| Polyphosphokinase (PPK2) | 10.2 |

### Example 14: Enzymatic Production of Phosphocreatine (Without PAP)

According to the preparation methods of Examples 1 and 3-5, the genes of creatine kinase (CK), adenosine kinase (AK), adenylate kinase (ADK), polyphosphokinase (PPK) and the recombinant enzyme overexpressed in E. coli cells respectively were obtained and fermented. According to the method described in Example 3 of Chinese Patent CN1982445B, the mixed immobilized cells of the recombinant enzyme overexpressed in E. coli cells of creatine kinase, the recombinant enzyme overexpressed in E. coli cells of adenosine kinase, the recombinant enzyme overexpressed in E. coli cells of adenylate kinase and the recombinant enzyme overexpressed in E. coli cells of polyphosphokinase were prepared on a carrier. Table 6 below shows the wet weight of the respective cells after centrifugation.

The mixing weight ratio of the respective cells was substantially based on the corresponding enzyme activity. The shape of the carrier was a bar as follows: 25 cm long, 5 cm wide, and 5 mm thick, with an actual weight of 34.5 g. The substrates contained in the reaction solution were as follows: 8.94 g/L of creatine monohydrate, 3 g/L of adenosine triphosphate disodium salt, 16.4 g/L of magnesium chloride hexahydrate, 6 g/L of adenosine, and 13.3 g/L of polyphosphoric acid. After adding the above substrates, the resultant was stirred and dissolved with 1 liter of deionized water at about 45° C. until the reaction solution was clear, and the pH was adjusted to 8.5 with 2 M sodium hydroxide solution. 300 ml of the above reaction solution was added to the reaction control tank, with the temperature being 37° C., the pH being adjusted to 8.35-8.60, and the flow rate of the high-flow water pump set at 3 L/min. The reaction continued for 60 minutes. During the reaction, the content of adenosine triphosphate (ATP), adenosine monophosphate (AMP) and phosphocreatine continued to increase. During the reaction, adenosine triphosphate (ATP), adenosine diphosphate (ADP), and adenosine monophosphate (AMP) were slowly degraded to adenosine over time course under the influence of miscellaneous enzymes, but adenosine monophosphate (AMP) has failed to regenerate due to the lack of polyphosphate:AMP phosphotransferase ,which ultimately leads to the termination of regeneration into adenosine diphosphate (ADP) and adenosine triphosphate (ATP). Thus, a portion of adenosine monophosphate (AMP), a small amount of adenosine diphosphate (ADP), adenosine triphosphate (ATP), adenosine and a large amount of adenine remained at the end of the reaction; and the concentration of phosphocreatine product solution was 4.8 mM, with a total weight of 0.3 g.

TABLE 6

| Enzyme | Weight of cells (g) |
|---|---|
| Creatine kinase (CK) | 9.1 |
| Adenosine kinase (AK) | 8.3 |
| Adenylate Kinase (ADK) | 10 |
| Polyphosphokinase (PPK2) | 7.5 |

### Example 15: Enzymatic Production of Phosphocreatine (Without PPK)

According to the preparation methods of Examples 1-3 and 5, the genes of creatine kinase (CK), adenosine kinase (AK), adenylate kinase (ADK), polyphosphate:AMP phosphotransferase (PAP) and the recombinant enzyme overexpressed in E. coli cells respectively were obtained and fermented. According to the method described in Example 3 of Chinese Patent CN1982445B, the mixed immobilized cells of the recombinant enzyme overexpressed in E. coli cells of creatine kinase, the recombinant enzyme overexpressed in E. coli cells of adenosine kinase, the recombinant enzyme overexpressed in E. coli cells of adenylate kinase and the recombinant enzyme overexpressed in E. coli cells of polyphosphate:AMP phosphotransferase were prepared on a carrier. Table 7 below shows the wet weight of the respective cells after centrifugation. The shape of the carrier was a bar as follows: 23 cm long, 5 cm wide, and 5 mm thick, with an actual weight of 32.8 g. The substrates contained in the reaction solution were as follows: 8.94 g/L of creatine monohydrate, 3 g/L of adenosine triphosphate disodium salt, 16.4 g/L of magnesium chloride hexahydrate, 6 g/L of adenosine, and 13.3 g/L of polyphosphoric acid. After adding the above substrates, the resultant was stirred and dissolved with 1 liter of deionized water at about 45° C. until the reaction solution was clear, and the pH was adjusted to 8.5 with 2 M sodium hydroxide solution. The reaction solution was ready for use when the temperature thereof dropped to room temperature. 300 ml of the above reaction solution was added to the reaction control tank, with the temperature being 37° C., the pH being adjusted to 8.35-8.60, and the flow rate of the high-flow water pump set at 3 L/min. The reaction continued for 60 minutes. During the reaction, adenosine monophosphate (AMP), adenosine triphosphate (ATP) and phosphocreatine continued to increase. At the end of the reaction, adenosine monophosphate (AMP), adenosine diphosphate (ADP) and adenosine remained; and the concentration of the phosphocreatine product was 4.4 mM, with a total weight of 0.28 g.

TABLE 7

| Enzyme | Weight of cells (g) |
|---|---|
| Creatine kinase (CK) | 9.0 |
| Adenosine kinase (AK) | 8.5 |
| Adenylate Kinase (ADK) | 10 |
| Polyphosphate: AMP | 7.8 |
| phosphotransferase (PAP) | |

### Example 16: Enzymatic Production of Phosphocreatine (Without PPK2 and ADK)

According to the preparation methods of Examples 1-2 and 5, the genes of creatine kinase (CK), polyphosphate:AMP phosphotransferase (PAP), adenosine kinase (AK) and the recombinant enzyme overexpressed in E. coli cells expressing the genes respectively were obtained and fermented. According to the method described in Example 3 of Chinese Patent CN1982445B, the mixed immobilized cells of the E. coli cells expressing creatine kinase, the E. coli cells expressing polyphosphate:AMP phosphotransferase, and the E. coli cells expressing adenosine kinase were prepared on a carrier. Table 8 below shows the wet weight of the respective cells after centrifugation. The shape of the carrier was a bar as follows: 24 cm long, 5 cm wide, and 5 mm thick, with an actual weight of 34.2 g. The substrates contained in the reaction solution were as follows: 8.94 g/L of creatine monohydrate, 3 g/L of adenosine triphosphate disodium salt, 16.4 g/L of magnesium chloride hexahydrate, 6 g/L of adenosine, and 13.3 g/L of polyphosphoric acid. After adding the above substrates, the resultant was stirred and dissolved with 1 liter of deionized water at about 45° C. until the reaction solution was clear, and the pH was adjusted to 8.5 with 2 M sodium hydroxide solution. 300 ml of the above reaction solution was added to the reaction control tank, with the temperature being 37° C., the pH being adjusted to 8.35-8.60, and the flow rate of the high-flow water pump set at 3 L/min. The reaction continued for 60 minutes. Due to the lack of PPK2 and ADK in the combination of adenosine triphosphate (ATP) enzymatic regeneration and production reaction, the reaction failed to regenerate adenosine triphosphate (ATP) from adenosine diphosphate (ADP); and the amount of adenosine diphosphate (ADP) was constantly increasing during the reaction. At the end of the reaction, a large amount of adenosine diphosphate (ADP), a small amount of adenosine monophosphate (AMP) and adenosine remained; and the concentration of the Phosphocreatine product solution was 0.9 mM, with a total weight of 0.06 g.

TABLE 8

| Enzyme | Weight of cells (g) |
|---|---|
| Creatine kinase (CK) | 9.0 |
| Adenosine kinase (AK) | 8.5 |
| Polyphosphate: AMP | 7.5 |
| phosphotransferase (PAP) | |

The present invention is not limited by the above specific description, and various modifications or changes can be made to the present invention within the scope generalized by the claims. These modifications or changes all fall within the scope of protection of the present invention.

1. A method for additionally increasing an amount of adenosine triphosphate (ATP) and regenerating ATP in an enzymatic reaction, wherein a reaction substrate of the enzymatic reaction comprises adenosine triphosphate (ATP) or a salt thereof and the method includes the following steps:
a first enzyme or enzyme group for producing adenosine monophosphate (AMP) during the enzymatic reaction to additionally increase the amount of adenosine triphosphate (ATP), and
adding a second enzyme or enzyme group responsible for adenosine triphosphate (ATP) regeneration at the same time as adding the first enzyme or enzyme group.

2. (canceled)

3. The method according to claim 1, wherein a third enzyme or enzyme group is added at the same time as, before or after adding the first enzyme or enzyme group and adding the second enzyme or enzyme group.

4. The method according to claim 1, wherein the first enzyme or enzyme group comprises adenosine kinase (AK).

5. The method according to claim 1, wherein the reaction substrate further comprises at least one of polyphosphoric acid or a salt thereof and an auxiliary ion, and
wherein the auxiliary ion is preferably at least one of magnesium ion, sodium ion, potassium ion and chloride ion, more preferably at least one of magnesium ion and potassium ion; and the auxiliary ion may be in the form of an inorganic salt or an organic salt, preferably at least one of magnesium chloride hexahydrate, sodium chloride, manganese chloride, magnesium sulfate, and potassium carbonate, and more preferably at least one of magnesium chloride hexahydrate, sodium chloride, and potassium carbonate.

6. The method according to claim 3, wherein the second enzyme or enzyme group comprises at least one selected from a group consisting of polyphosphate:AMP phosphotransferase (PAP), polyphosphokinase (PPK) and adenylate kinase (ADK); and
the third enzyme or enzyme group comprises at least one selected from a group consisting of creatine kinase (CK), glutamate kinase (GK), nicotinamide adenine dinucleotide kinase (NK), and other enzymes or enzyme groups for performing phosphorylation, phosphate group transfer or polypeptide synthesis of amino acids, peptides or proteins in which adenosine triphosphate (ATP) serves as one of the substrates.

7. The method according to claim 3, wherein the enzymatic reaction comprises at least one selected from a group consisting of:
(i) the first enzyme or enzyme group comprises adenosine kinase (AK), the reaction substrate comprises adenosine, polyphosphoric acid and adenosine triphosphate (ATP), and the reaction product comprises adenosine monophosphate (AMP) and adenosine diphosphate (ADP);
(ii) the first enzyme or enzyme group comprises adenosine kinase (AK), and the second enzyme or enzyme group comprises polyphosphate:AMP phosphotransferase (PAP), and the reaction substrate comprises adenosine monophosphate (AMP) and polyphosphoric acid, and the reaction product contains adenosine diphosphate (ADP) and polyphosphoric acid;
(iii) the first enzyme or enzyme group comprises adenosine kinase (AK), and the second enzyme or enzyme group comprises adenylate kinase (ADK), and the reaction substrate comprises adenosine diphosphate (ADP) and polyphosphoric acid, and the reaction product contains adenosine monophosphate (AMP) and adenosine triphosphate (ATP);
(iv) the first enzyme or enzyme group comprises adenosine kinase (AK), and the second enzyme or enzyme group comprises polyphosphokinase (PPK), and the reaction substrate comprises adenosine diphosphate (ADP) and polyphosphoric acid, and the reaction product contains adenosine triphosphate (ATP) and polyphosphoric acid; and
(v) the first enzyme or enzyme group comprises adenosine kinase (AK), and the second enzyme or enzyme group comprises polyphosphate:AMP phosphotransferase (PAP), polyphosphokinase (PPK) and adenylate kinase (ADK), and the third enzyme or enzyme group comprises creatine kinase, the reaction substrate comprises creatine and adenosine triphosphate (ATP), and the reaction product comprises phosphocreatine, adenosine diphosphate (ADP) and polyphosphoric acid;
(vi) use of all or at least one of polyphosphate:AMP phosphotransferase (PAP), adenylate kinase (ADK) and polyphosphokinase (PPK), and adenosine kinase (AK), with adenosine triphosphate (ATP) as the reaction substrate, to carry out reaction simultaneously in the same reaction system in the manner of mixing, parallel or series, or carry out reaction separately in different reaction systems;
(vii) only use of all or at least one of polyphosphate:AMP phosphotransferase (PAP), adenosine kinase (AK) and polyphosphokinase (PPK), with adenosine triphosphate (ATP) as the reaction substrate, to carry out reaction simultaneously in the same reaction systems in the manner of mixing, parallel or series, or carry out reaction separately in different reaction systems; and
(viii) only use of adenosine kinase, with adenosine as a substrate, to produce adenosine monophosphate (AMP), thereby newly adding adenosine triphosphate (ATP).

8. The method according to claim 1, wherein the second enzyme or enzyme group regenerates adenosine diphosphate (ADP) and adenosine triphosphate (ATP) from adenosine monophosphate (AMP) and adenosine diphosphate (ADP), respectively; and
the first enzyme or enzyme group synthesizes adenosine monophosphate (AMP) from adenosine.

9. The method according to claim 1, wherein the enzymatic reaction comprises synthesizing adenosine monophosphate (AMP) using adenosine and adenosine triphosphate (ATP) as substrates.

10. The method according to claim 1, wherein the reaction substrate further comprises at least one selected from a group consisting of creatine monohydrate, sodium glutamate and nicotinamide adenine dinucleotide.

11. The method according to claim 1, wherein the first enzyme or enzyme group to be added is determined according to the level of the degradation product of adenosine triphosphate (ATP) produced in the enzymatic reaction, wherein the degradation product is preferably adenosine diphosphate (ADP), adenosine monophosphate (AMP) and/or adenosine.

12. The method according to claim 3, wherein the first enzyme or enzyme group, or the second enzyme or enzyme group, or the third enzyme or enzyme group is added in the form of purified or non-purified cell lysates, liquid enzymes, immobilized cells or immobilized enzymes.

13. The method according to claim 1, wherein the conditions at which the enzymatic reaction is performed are: a temperature of 28-40 degrees Celsius, preferably 30-38 degrees Celsius, and more preferably 33-37 degrees Celsius; and a pH value of 5-9, preferably 6-8.5, and more preferably 7-7.75.

14. An enzymatic reaction composition comprising a substrate, a first enzyme or enzyme group for producing adenosine monophosphate (AMP) and a second enzyme or enzyme group responsible for adenosine triphosphate (ATP) regeneration, wherein the substrate comprises adenosine triphosphate (ATP) or a salt thereof, and adenosine.

15. The enzymatic reaction composition according to claim 14, wherein the substrate further comprises at least one selected from a group consisting of polyphosphoric acid or a salt thereof, an auxiliary ion, and creatine monohydrate;
wherein the auxiliary ion is preferably at least one of magnesium ion, sodium ion, potassium ion and chloride ion, and more preferably at least one of magnesium ion and potassium ion; the auxiliary ion may be in the form of an inorganic salt or an organic salt, preferably at least one of magnesium chloride hexahydrate, sodium chloride, manganese chloride, magnesium sulfate, and potassium carbonate, and more preferably at least one of magnesium chloride hexahydrate, sodium chloride, and potassium carbonate.

16. The enzymatic reaction composition according to claim 14, wherein the enzymatic reaction composition further comprises a third enzyme or enzyme group.

17. The enzymatic reaction composition according to claim 16, wherein the first enzyme or enzyme group comprises adenosine kinase (AK),
preferably, the enzymatic reaction composition further comprises the third enzyme or enzyme group, wherein the third enzyme or enzyme group comprises creatine kinase (CK), glutamate kinase (GK), nicotinamide adenine dinucleotide kinase (NK), or other enzymes or enzyme groups for performing phosphorylation, phosphate group transfer or polypeptide synthesis of amino acids, peptides or proteins in which adenosine triphosphate (ATP) serves as one of the enzymatic reaction substrates, and
the second enzyme or enzyme group comprises at least one selected from consisting of polyphosphate:AMP phosphotransferase (PAP), polyphosphate kinase (PPK), and adenylate kinase (ADK).

18. The enzymatic reaction composition according to claim 16, wherein the first enzyme or enzyme group, the second enzyme or enzyme group and the third enzyme or enzyme group are contained in the enzymatic reaction composition in the form of purified or non-purified cell lysates, liquid enzymes, immobilized cells or immobilized enzymes.

19. The enzymatic reaction composition according to claim 17, wherein the polyphosphate:AMP phosphotransferase (PAP), adenosine kinase (AK), polyphosphokinase (PPK) and adenylate kinase (ADK), creatine kinase (CK), glutamate kinase (GK) and nicotinamide adenine dinucleotide (NK) are each respectively recombinant enzyme, and are expressed in E. coli separately or in combination.

20. A method for phosphorylation or phosphate group transfer of amino acids, nucleic acids, peptides or proteins using adenosine triphosphate (ATP) as a substrate, including the method of claim 1.

21. The method according to claim 20, wherein the amino acid, nucleic acid, peptide or protein is at least one of creatine, glutamic acid, and nicotinamide adenine dincleotide.


SEQUENCE LISTING (21 sequences)

SEQ ID NO: 1 - DNA, 36 bp, Artificial Sequence
  CPK1 Primer
ctgaccggatccatgccgttcggtaacacccacaac

SEQ ID NO: 2 - DNA, 36 bp, Artificial Sequence
  CPK2 Primer
tatgcggaattcttacttctgggcggggatcatgtc

SEQ ID NO: 3 - DNA, 36 bp, Artificial Sequence
  PAP1 Primer
ctgaccggatccatgttcgaatccgcggaagttggc

SEQ ID NO: 4 - DNA, 39 bp, Artificial Sequence
  PAP2 Primer
tatgcgaagcttttacttgtccttcttgtacgccgcctc

SEQ ID NO: 5 - DNA, 36 bp, Artificial Sequence
  AK1 Primer
ctgaccggatccatggcagtcgattcctccaactcg

SEQ ID NO: 6 - DNA, 36 bp, Artificial Sequence
  AK2 Primer
tatgcggaattcttaacacggaagtgaagtgaagct

SEQ ID NO: 7 - DNA, 36 bp, Artificial Sequence
  PPK-1 Primer
ctgaccggatccatgagcaagtccgacgacgacgag

SEQ ID NO: 8 - DNA, 36 bp, Artificial Sequence
  PPK-2 Primer
tatgcggaattcttaccgcgccaaccgcccatcttc

SEQ ID NO: 9 - DNA, 36 bp, Artificial Sequence
  ADK1 Primer
ctgaccggatccatgaatatcattttgatgggttta

SEQ ID NO: 10 - DNA, 36 bp, Artificial Sequence
  ADK2 Primer
tatgcggaattcttacaaatgatctaaaatatcaat

SEQ ID NO: 11 - DNA, 6090 bp, Artificial Sequence
  pGEX-2T(+)-CK
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccatgccgttcggtaacacccacaacaagcacaagctgaacttcaaggctgaggaggaatacccagacctcagcaagcacaacaaccacatggccaaggcgctgaccctcgagatttacaagaagctgcgggacaaggagacgccctctggcttcactctggatgacgtcatccagaccggtgtggacaacccaggtcaccccttcatcatgaccgtgggctgtgtggctggtgatgaggagtcctatacggttttcaaggacctctttgaccccatcatccaggaccggcacgggggcttcaaacccacagacaagcacaagactgacctcaaccatgagaacctcaagggtggagatgatctggaccccaactacgtgctcagcagccgtgtccgcacgggccgcagcatcaagggctatgcactgcctccccactgctcccgcggcgagcgccgggccgtggagaaactctccgtggaagccctcaacagcctgacgggtgagttcaaggggaaatactaccctctgaagagcatgacagagcaggagcagcagcagctcatcgacgaccacttcctgtttgacaagcccgtgtccccgctgctgctggcttcaggcatggcccgagactggcccgatgcccgtggcatctggcacaatgacaacaagagcttcctggtgtgggtgaacgaggaggatcacctccgagtcatctccatggagaaggggggcaacatgaaggaggttttccgccgcttctgcgtggggctgcagaagattgaggagatattcaagaaagccggccacccgttcatgtggaacgagcacctgggctacgtgctcacctgcccatctaacctgggcaccgggctgcgtggaggcgtgcatgtcaagttggcgcacttgagcaagcatcccaagttcgaggagatcctcactcgcctgcgcctgcagaagcgaggcacaggtggtgtggacacggctgccgtgggctcagtgttcgacgtgtccaacgccgatcggctgggctcgtcggaggtagaacaggtgcagctggtggtggatggtgtgaagctcatggtggagatggagaagaagctggagaagggccagtccatcgacgacatgatccccgcccagaagtaagaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 12 - DNA, 6435 bp, Artificial Sequence
  pGEX-2T(+)-PAP
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccatgttcgaatccgcggaagttggccacagcatcgacaaggacacctacgagaaggccgtcatcgagttgcgcgaagcgctgctcgaggcgcagttcgagctcaagcagcaggcgcgcttcccggtgatcatcctgatcaacggcatcgagggcgccggcaagggcgagacggtcaagctgctcaacgagtggatggacccgcgcctaatcgaggtgcagagcttcctccgtccttccgacgaggagctggagcggccgccgcagtggcgcttctggcggcgcctgccgcccaaggggcggaccggtatcttcttcggcaactggtacagccagatgctctacgcgcgggtcgaggggcatatcaaggaggccaagctggaccaggccatcgatgccgccgaacgcttcgagcgcatgctctgcgacgaaggcgcgctgctcttcaagttctggttccatctctccaagaaacagttgaaggagcgtctcaaggcgctggagaaggacccgcagcacagttggaagctcagtccgctggactggaagcagagcgaggtctacgaccgcttcgtgcattacggcgagcgtgtgctgcgccgtaccagccgggactacgcgccctggtacgtggtggaaggcgcggacgagcgctaccgcgccctgaccgtcggccgcatccttctcgaagggttgcaggccgcgctggccaccaaggagcgcgccaagcgccagccgcacgccgcaccgctggtgtcgagcctggacaaccgtggcctgctggactccctggacctgggccagtacctggacaaggacgcctacaaggagcagctcgccgccgagcaggcgcgcctggccgggctgatccgcgacaagcgcttccgccagcattcgctggtcgcggtgttcgagggcaacgacgcggccggcaagggcggcgccatccgccgtgtcaccgacgcgctggacccgcgccagtaccatatcgtgccgatcgccgcgccgaccgaagaggagcgtgcgcagccctatctctggcgcttctggcggcacattccggcgcgtcgccagttcaccatcttcgaccgttcctggtacggccgcgtgctggtggagcgcatcgagggcttctgcgcaccggccgactggctacgcgcctatggcgagatcaatgacttcgaggagcagctcagcgagtacgggatcatcgtggtgaagttctggctggcgatcgacaagcagacccagatggagcgcttcaaggaacgcgagaaaaccccctacaagcgctacaagatcaccgaggaagactggcgcaaccgcgacaagtgggaccagtacgtggacgcggtgggcgatatggtcgaccgtaccagcaccgagatcgcgccctggaccctggtcgaagccaacgacaagcgcttcgcccgggtcaaggtgctgcgcaccatcaacgacgccatcgaggcggcgtacaagaaggacaagtaagaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 13 - DNA, 5592 bp, Artificial Sequence
  pGEX-2T(+)-ADK
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccatgaatatcattttgatgggtttacctggcgcaggtaaaggaactcaagcaagtgaaattgtcaagaaattcccaataccccacatttcaactggtgacatgttcagaaaagctataaaagaagaaactgaattaggtaaagaagctaagtcttatatggaccgtggcgaattagttcctgatgaagtgactgtaggtatcgttaaggaaagaatttctgaagacgatgcaaaaaaaggctttttattagatggcttcccaagaacaatcgagcaagctgaggcattaaataatattatgtctgagcttgacagaaacattgatgctgtcatcaatatcgaagttccggaagaagaattaatgaaccgtcttacaggtcgtcgaatctgtgagtcatgtggtacaacgtatcatcttgtatttaatcctccgaaggtcgaaggtatttgtgatatcgatggtggtaaattgtatcaacgagaagatgataatcctgaaacggtagctaatcgtttgagtgttaatattaaacaatctaagcctattttagatttctatgatcaaaaaggtgtattgaaaaatattgatggttcaaaagatattagcgacgttaccaaagatgtcattgatattttagatcatttgtaagaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 14 - DNA, 5706 bp, Artificial Sequence
  pGEX-2T(+)-PPK
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccatgagcaagtccgacgacgacgaggccgaactcgtccagctgcaactggcgctgatcgccctgcagaaaaaggccatcaaggacggcgacaagatcctggtggtcttcgagggccgcgacgccgccggcaaggacggcgtcatcgcacggatcaccgagcacctgtcgcggcgcgccaccacggtcgtggccctgcccaagcccaccgaccgcgagcgcagcgagtggtacttccagcgctatgtcgaatggctgccggcctgcggcgaggcggtgctgttcaaccgctcctggtacaatcgggccggcgtcgagcgggtgatggatttctcgacgccgcagcagcaggagcagttcctgcgcgacgtcccggccttcgagcggatgctggtcgagaacggcatgcgctatgtaaagttctggctggacatcagccgcgaggagcaggccaagcgcctcaaatcccgccgcgaagacccgctgaaggccttcaagaccagtccgctggacgccgtcgcccaggagaagtgggacgactacaccaaggcccgcgacgagatgctgatgcgcacccacagcgacatcgcgccctggatctgcgtccgcgccgaccacaagaaggccgcccggctgaacgtgatccgctggctgctgcacgcggccggcgacaagaagatcctcaagggcgtggagaggcccgatcctgcggtgatcttcccgttcgagccggcggcgctggaagatgggcggttggcgcggtaagaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 15 - DNA, 6036 bp, Artificial Sequence
  pGEX-2T(+)-CK
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccatggcagtcgattcctccaactcggcgacgggccccatgagagtgttcgcgatagggaatccgattctggacctcgtcgccgaggtgccctcttccttcctcgacgagttcttcctcaagaggggagacgcgactctcgcgactccggaacaaatgcgcatctactccactcttgaccagttcaatccgacttccctgcctggtggcagtgcactcaactctgttcgtgtcgtccagaaactcttgagaaagcctggcagcgctgggtacatgggagccattggcgatgacccccgtggccaggtgctaaaagaactctgcgacaaggaaggcctcgccactcgcttcatggtcgctccaggtcagagcaccggagtgtgtgccgtgttgatcaacgagaaagaacggacgctctgcacgcatctcggcgcatgcggttctttccgactgcctgaggactggactaccttcgcttccggagcgctcatcttttatgccaccgcctacacacttactgcgaccccaaagaacgcgcttgaggtagcggggtatgctcatggcataccgaacgccatctttacgctgaacttgtcggctccgttctgcgttgagctgtacaaggacgcgatgcagtctttgcttctccatacgaacattctctttggaaacgaagaggagttcgcgcatttggcgaaagttcacaatctcgtggctgctgagaagacggcgctgtccactgcgaacaaagaacacgcagtggaagtgtgcacgggcgccttgcgtctgctcactgccggtcagaacacgggcgcgacgaagctcgttgtcatgacacggggtcacaaccccgtcattgctgctgagcaaacagccgacggaactgtggtcgtccacgaagttggtgtcccggtggttgctgcggaaaaaattgtggacaccaacggcgcgggcgacgcgtttgtcggcggttttctctatgcactctcgcagggaaaaacggtgaaacagtgcatcatgtgcggcaacgcttgtgctcaggatgtgattcagcatgtaggatttagtctcagcttcacttcacttccgtgttaagaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 16 - DNA, 36 bp, Artificial Sequence
  GK1 Primer
ctgaccggatccatgcgggacaaggtgactggcgcg

SEQ ID NO: 17 - DNA, 36 bp, Artificial Sequence
  GK2 PRimer
tatgcggaattcttagaccagaaccagattgtcgcg

SEQ ID NO: 18 - DNA, 36 bp, Artificial Sequence; identical to SEQ ID NO: 16
  NK1 Primer

SEQ ID NO: 19 - DNA, 36 bp, Artificial Sequence; identical to SEQ ID NO: 17
  NK2 Primer

SEQ ID NO: 20 - DNA, 6063 bp, Artificial Sequence
  pGEX-2T(+)-GK
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccatgcgggacaaggtgactggcgcgcggcgctgggtggtgaagatcggtagcgccctgctgacggccgatggccgtgggctggatcgcaatgccatggccgtttgggtcgagcagatggtggcgctgcattgcgccggcatcgagctggtgctggtgtcgtccggcgccgtcgccgccggtatgagccgcctgggctgggtgtcccgacctagcgcgatgcacgaactccaggccgccgcctcggtggggcaaatggggttggtgcaggcctgggagtccagcttcgccctgcatggcctgcagaccgcccaggtgttgctgacccatgacgacctctccgaccgcaagcgctacctgaacgcccgcagcaccctgcggaccctggtcgagctgggcgtggtcccggtgatcaacgagaacgacacggtggtcaccgacgagatccgcttcggcgacaacgacaccctggcggcactggtggccaacctggtcgaggccgacctgctggtgatccttaccgatcgcgacgggatgttcgacgccgatccgcgcaacaatcccgatgcccaactgatctacgaggcgcgtgccgatgatccgcagctcgatgcggtggccggcggcagtgccggcgccctggggtgcggcggaatgcagaccaagctgcgggcggcgcgcctggcggcgcgttccggcgggcatacggtgatcgtcggcgggcgtatcgagcgtgtcctcgaccgcctgcgcgccggcgaacgcctcggcaccctgttgacccctgatcgcagtcgcaaggcggcgcgcaagcaatggctggccggccacttgcagatgcgcggcaccctggtgctggacgatggggcggtgaaggcggtgagccaggatcacaagagcctgttgccggttggggtcaaggcggtccagggcagcttccgtcgcggcgagatggtggtctgtgtggaccagggcgggcgcgaagtggcgcgcggcctggtcaactacagtgcgctggaggcgcagaagatcctcggtcagccgacggatgccatcgaggccttgctcggctacgtggatgggccggagctggtgcatcgcgacaatctggttctggtctaagaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 21 - DNA, 5868 bp, Artificial Sequence
  pGEX-2T(+)-NK
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccatgaccgctcatcgcagtgttctgctggtcgtccacaccgggcgcgacgaagccaccgagaccgcacggcgcgtagaaaaagtattgggcgacaataaaattgcgcttcgcgtgctctcggccgaagcagtcgaccgagggtcgttgcatctggctcccgacgacatgcgggccatgggcgtcgagatcgaggtggttgacgcggaccagcacgcagccgacggctgcgaactggtgctggttttgggcggcgatggcacctttttgcgggcagccgagctggcccgcaacgccagcattccggtgttgggcgtcaatctgggccgcatcggctttttggccgaggccgaggcggaggcaatcgacgcggtgctcgagcatgttgtcgcacaggattaccgggtggaagaccgcttgactctggatgtcgtggtgcgccagggcgggcgcatcgtcaaccggggttgggcgctcaacgaagtcagtctggaaaagggcccgaggctcggcgtgcttggggtggtcgtggaaattgacggtcggccggtgtcggcgtttggctgcgacggggtgttggtgtccacgccgaccggatcaaccgcctatgcattctcggcgggaggcccggtgctgtggcccgacctcgaagcgatcctggtggtccccaacaacgctcacgcgctgtttggccggccgatggtcaccagccccgaagccaccatcgccatcgaaatagaggccgacgggcatgacgccttggtgttctgcgacggtcgccgcgaaatgctgataccggccggcagcagactcgaggtcacccgctgtgtcacgtccgtcaaatgggcacggctggacagtgcgccattcaccgaccggctggtgcgcaagttccggttgccggtgaccggttggcgcggaaagtaagaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt